(19) United States
(12) Patent Application Publication
(10) Pub. No.: US 2012/0219526 A1
(43) Pub. Date: Aug. 30, 2012

(54) NUTRITIONAL COMPOSITIONS COMPRISING LACTOFERRIN AND PROBIOTICS AND KITS OF PARTS THEREOF

(75) Inventors: Petra Gerda Klassen, St. Legier (CH); Corinne Magliola, Pully (CH); Daniel Roland Manser, Spiez (CH); Theresa Voss, Minnetonka, MN (US)

(73) Assignee: Nestec S.A., Vevey (CH)

(21) Appl. No.: 13/505,197

(22) PCT Filed: Oct. 29, 2010
(86) PCT No.: PCT/EP10/66543

§ 371 Date: Apr. 30, 2012

(30) Foreign Application Priority Data
Oct. 29, 2009 (EP) 09174469.8

(51) Int. Cl. A61K 35/74; A61P 3/02; A61P 1/00; A23L 1/48

(52) U.S. Cl. 424/93.3; 426/61; 426/2; 424/93.45; 424/93.44; 424/93.4; 424/93.48; 424/93.43

(57) ABSTRACT

The present invention relates to nutritional composition for infants and/or children comprising lactoferrin and probiotics. These compositions have been found to be useful in providing health benefits in infants and/or children. A method of manufacture of these compositions is also part of the invention. These compositions may be in the form of a kit of parts wherein a first composition according to the invention and a second composition according to the invention are each adapted to fulfil the nutritional requirements in two different age groups.

### FIELD OF THE INVENTION

The present invention relates to nutritional composition for infants and/or children comprising lactoferrin and probiotics. These compositions have been found to be useful in providing health benefits in infants and/or children. A method of manufacture of these compositions is also part of the invention. These compositions may be in the form of a kit of parts wherein a first composition according to the invention and a second composition according to the invention are each adapted to fulfil the nutritional requirements in two different age groups.

### BACKGROUND OF THE INVENTION

Lactoferrin is a major component of human breast milk. It is considered to have a range of biological functions in infants, including roles in gut maturation, immune development, prevention of infections and iron absorption. Lactoferrin is present at very high levels in human colostrum (up to 10 g/l has been reported), with levels in mature human milk decreasing significantly as the infant ages (2-3 g/L at 1 month, 1 g/l at 6 months). Lactoferrin has been of interest for use in infant formula for some time but the high cost has generally prevented its use.

The use of lactoferrin in infant formula is known from EP 0 295 009 B1. Therein lactoferrin is used as a dietary ingredient for promoting the growth of the gastrointestinal tract.

Other ingredients to promote gut health such as probiotics have also recently been added to infant formula. For instance WO 2006/108824, WO 2004/112508 and WO 2008/056983 all describe infant formula comprising probiotics.

Probiotics are generally added to infant formula to provide benefits ranging from controlling or preventing colic, reducing regurgitations, improving gut motility, reducing allergies, improving immunity, etc. The benefits therefore span a wide variety of areas.

There is therefore a need to provide compositions which provide specific health benefits for a particular targeted population.

### OBJECT OF THE INVENTION

The object of the invention is therefore to provide a nutritional composition which induces particular health benefits and is adapted to a particular targeted population.

### SUMMARY OF THE INVENTION

This object is solved by means of the independent claims. The dependent claims further develop the central idea of the invention.

Thus, in a first aspect, the invention relates to a nutritional composition for infants and/or children comprising lactoferrin and probiotics.

A second aspect of the invention relates to a nutritional composition according to any of claims 1 to 8 for use in fulfilling at least partially the nutritional requirements of infants and/or children.

In a third aspect, a nutritional composition according to any of claims 1 to 8 for use in providing health benefits in infants and/or children is provided.

A method for preparing a nutritional composition comprising lactoferrin and probiotics comprising the steps of described also forms part of the present invention.

Finally, a kit of parts comprising a first composition according to any of claims 1 to 8 and a second composition according to any of claims 1 to 8 wherein said first composition and said second composition differ by at least the presence of one nutrient or by the concentration of said nutrient in said composition, characterised in that said first and second compositions are each adapted to fulfil the nutritional requirements in two different age groups, is also part of the present invention.

### DETAILED DESCRIPTION OF THE INVENTION

The invention relates to a nutritional composition for infants and/or children.

In one embodiment the composition of the invention is a complete nutritional composition, i.e. it is intended to provide all caloric and nutrient intake for the target infants. In one embodiment the composition is an infant formula, a started infant formula or a follow-up infant formula. In one embodiment the composition is in a dry powder form, intended to be reconstituted with water into a liquid nutritional composition. In another embodiment the composition is a cow milk-derived composition, preferably liquid, intended for infants and children between 1 and 3, between 1 and 6 years or between 3 and 6 years (so called “grown-up milk”). The composition of the invention, especially those intended for children between 1 and 6 or 3 and 6 years, can comprise cereals.

By “infants” is meant newborns from the age of 0 months to 24 months. Children are understood in the present invention to be from birth 0 to 6 years.

Preferably, the nutritional composition is adapted for infants and/or children who have an age of 0 months to 6 years, preferably 0 months to 3 years. In a preferred embodiment, the nutritional composition is adapted for infants and/or children of 0 months to 1 year or 1 year to 3 years.

The nutritional composition of the invention comprises lactoferrin and probiotics.

A probiotic may be defined as live microorganisms which, when administered in adequate amounts, confer a health benefit on the host. It may be in the form of a live microbial feed supplement which beneficially affects the host animal by improving its intestinal microbial balance. The probiotic used in the present composition may be selected from the group comprising of Bifidobacterium, Lactobacillus, Streptococcus, Enterococcus and Saccharomyces or mixtures thereof, preferably selected from the group consisting of Bifidobacterium longum, Bifidobacterium lactis, Lactobacillus acidophilus, Lactobacillus rhamnosus, Lactobacillus paracasei, Lactobacillus johnsonii, Lactobacillus plantarum, Lactobacillus salivarius, Lactobacillus reuteri, Enterococcus faecium, Streptococcus sp. and Saccharomyces boulardii or mixtures thereof. More preferably the probiotic is selected from the group comprising of Lactobacillus rhamnosus CGMCC 1.3724 (nick name NCC4007 and LPR), Bifidobacterium lactis CNCM 1-3446 sold inter alia by the Christian Hansen company of Denmark under the trade mark Bb12 (nick mane NCC2818), Bifidobacterium longum ATCC BAA-999 sold by Morinaga Milk Industry Co. Ltd. of Japan under the trade mark BB536, Lactobacillus paracasei CNCM 1-2116 (nick name NCC2461 and ST11), Lactobacillus johnsonii CNCM 1-1225 (nick name NCC533 and La1), Lactobacillus fermentum VRI 003 sold by Probiomics (Australia), under the trademark PCC, Bifidobacterium longum CNCM 1-2170, Bifidobacterium longum CNCM 1-2618, Bifidobacterium breve sold by Danisco (Denmark) under the trade mark Bb-03, Bifidobacterium breve sold by Morinaga (Japan) under the trade mark M-16V and the strain of Bifidobacterium breve sold by Institut Rosell (Lallemand) (Canada) under the trade mark R0070, Lactobacillus paracasei CNCM 1-1292, Lactobacillus rhamnosus ATCC 53103 obtainable inter alia from Valio Oy of Finland under the trade mark LGG, Enterococcus faecium SF 68, and mixtures thereof. A preferred probiotic is Lactobacillus rhamnosus CGMCC 1.3724. Another preferred probiotics is Lactobacillus reuteri, especially Lactobacillus reuteri ATCC 55730, ATCC PTA 6475, ATCC PTA 4659 and ATCC PTA 5289, and more particularly Lactobacillus reuteri ATCC 55730 and L. reuteri DSM 17938 obtainable from Biogaia AB (Kungsbroplan 3A Stockholm, Sweden). It is foreseen that the composition of the present may comprise more than one probiotic, preferably targeting different health effects, and most preferably synergistically reinforcing the health effect(s). In one embodiment the probiotic is selected from the probiotics that are naturally found in human breast milk.

Preferably, the probiotic is present in the composition in an amount equivalent to between 10³ and 10¹⁰ cfu/g of dry composition (cfu=colony forming unit). This expression includes the possibilities that the bacteria are live, inactivated or dead or even present as fragments such as DNA or cell wall materials. In other words, the quantity of bacteria which the formula contains is expressed in terms of the colony forming ability of that quantity of bacteria as if all the bacteria were live irrespective of whether they are, in fact, live, inactivated or dead, fragmented or a mixture of any or all of these states.

Preferably the probiotic is present in an amount equivalent to between 10⁴ to 10⁹ cfu/g of composition, even more preferably in an amount equivalent to between 10⁶ and 10⁸ cfu/ g of composition.

In one embodiment the amount of probiotics present in the nutritional composition of the invention is low dose. By low dose is meant 10² to 10⁵ cfu/g of composition, preferably 10² to 10⁴ cfu/g of composition. It is anticipated that low dose of probiotics can, especially for very young infants, have a similar benefit as high dose of probiotics. Also low doses of probiotics may deliver specific benefits (such as improving gut comfort, stimulating and promoting gut maturation, and/or promoting immune maturation) that are masked or not present when the probiotic is administered at high dose.

The amount of lactoferrin in the present composition is preferably between 8 g and 0.1 g per liter of reconstituted nutritional composition (or per liter of ready-to-feed/ready-to-drink liquid composition). Such range can be between 3 g and 0.3 g per liter or between 1 g and 0.5 g per liter. In the powder form of the composition the amount of lactoferrin can be between 50 mg and 1500 mg per 100 g of dry composition (w/w). Such range can be between 100 mg and 1000 mg or between 200 mg and 800 mg per 100 g of dry composition (w/w). For premature, fragile, low weight infant or new born infants, it may be beneficial to have a relatively high amount of lactoferrin (for example between 8 g and 2 g per liter of ready-to-fed liquid composition or between 5 g and 3 g per liter. In a dry composition the ranges can be between 1500 mg and 500 mg or between 1000 mg and 750 mg per 100 g of dry composition). For relatively older infants (e.g. between 8 and 24 months) it may be beneficial to administer relatively lower dose of lactoferrin. For example the composition may comprise between 0.1 and 2 g, alternatively between 0.3 g and 1 g of lactoferrin per liter of ready-to-feed liquid composition or between 50 mg and 500 mg, alternatively between 100 mg and 300 mg of lactoferrin per 100 g of dry composition (w/w).

Lactoferrin can be human colostrum lactoferrin, human milk lactoferrin or bovine milk lactoferrin or lactoferrin of other source. A preferred source of lactoferrin is bovine milk lactoferrin that has been shown to provide the expected benefits when incorporated into the composition of the invention. The lactoferrin can be isolated from animal milk or can be a recombinant form of lactoferrin (such as recombinant human lactoferrin or recombinant bovine lactoferrin). The lactoferrin considered in the present invention can be pure isolated lactoferrin (or having a high degree of purity). In one embodiment the lactoferrin is comprised in a lactoferrin-rich fraction and is accompanied by other nutrients. The lactoferrin can be in a lactoferrin-rich fraction of bovine milk (by “rich” is meant that the content in lactoferrin is high than in the native ingredient). Lactoferrin can be sourced from DMV International (Netherlands), Murray Goulburn (Australia), □Tatua (New Zealand), □Fonterra (New Zealand), □Milei/Morinaga (Germany/Japan)

It has been found by the present inventors that a combination of probiotics and lactoferrin provides synergistic health benefits. Especially the benefits have been shown to be age-specific, with particular synergistic effect being more prominent for subgroups of infants of defined age. For example it has been found that the synergistic benefits (e.g. gut maturation, or immune maturation) may be of particular significance for premature, low weight at birth or otherwise fragile infants or in the very first few weeks of life (0 to 12 or 0 to 4 weeks). In older infants (e.g. 8 to 24 months)the beneficial synergistic effect may be significant in other health effects such as enhancing natural defenses, enhancing gut comfort or supporting growth.

Altogether these health benefits include promoting gut maturation, promoting the maturation of the gut nervous system, enhancing gut health, enhancing protection later in life, promoting the maturation of the immune system, contributing to support of natural defenses, contributing to support growth, enhancing gut comfort, reducing crying time, cramps and/or colics, fulfilling at least partially the nutritional requirements of said infant and/or child.

By “promoting gut maturation” is meant in particular (but not exclusively) maturation of the digestive system, including the related nervous system and immune system.

By “enhancing gut health” or by promoting “gut comfort” is meant in particular (but not exclusively) benefits selected from contributing to better balance the intestinal flora, reducing cramps, reducing colics, increasing gut absorption or selectivity of absorption.

By “enhancing protection later in life” is meant in particular (but not exclusively) reducing the risk of infections and/or allergies later in life. The long term effect of probiotics (for example for protection against infections or protection against atopic diseases)

By “contributing to support of natural defenses” is meant in particular (but not exclusively) enhancing the immune system, fighting infection, enhancing the maturation of the immune system.

By “contributing to support growth” is meant in particular (but not exclusively) enabling the growth of the infant or children to be as close as possible to the ideal growth curve.

Without being bound by the theory it is foreseen that lactoferrin can enhance the action of the probiotics by providing them with optimum conditions of growth and/or action. It is further hypothesized that the interaction between the probiotics, the lactoferrin and the host are key in the evidenced health effects. In particular the host —at particular age— can be receptive of stimuli of the combined probiotics and lactoferrin. Both together when administered at a defined age, the probiotics and the lactoferrin induce a response of the host. In turn, the response of the host induces a particular pattern of expression by the probiotics from which the host specifically benefits. Alternatively lactoferrin can act on the host and enable the host to be more sensitive to the action of the probiotics.

The compositions may further comprise additional ingredients selected from prebiotics, preferably N-acetylated oligosaccharide, sialylated oligosaccharide, galactooligosaccharides or mixtures thereof, vitamins, minerals, carbohydrates, fatty acids, lipids, protein, and any mixtures thereof.

Preferably, an embodiment of the prebiotic comprises an oligosaccharide produced from glucose, galactose, sialic acid, fucose, xylose, maltose, sucrose, lactose, starch, xylan, hemicellulose, inulin, or a mixture thereof. More preferably the oligosaccharide comprises fructooligosaccharide. Most preferably the prebiotic comprises a mixture of fructooligosaccharide and inulin. Preferably this mixture comprises PREBIO1® or a mixture of commercially available RAFTILOSE® and RAFTILINE®.

Preferably, an embodiment of the prebiotic comprises about 50% to about 90% fructooligosaccharide. More preferably it comprises about 60% to about 80% fructooligosaccharide.

Most preferably it comprises about 70% fructooligosaccharide.

Preferably, an embodiment of the prebiotic comprises about 10% to about 50% inulin. More preferably it comprises about 20% to about 40% inulin. Most preferably it comprises about 30% inulin.

The compositions may be in the form of powder or a liquid.

Preferably, the compositions of the invention have a caloric density of between 55 to 75 kcal, or between 60 and 70 kcal, per 100 ml of composition (ready-to-feed liquid composition or dry powder reconstituted for feeding), preferably 61 and 65 kcal per 100 ml of composition.

The caloric density can be adapted to match the best requirements for the age of the target infants or children: In one embodiment the caloric density is between and 67 kcal /100 ml for infants between birth and 2 months, between 61 and 65 kcal/100 ml for infants between 3 months and 12 months, and below 63 kcal/100 ml for children old than 12 months.

The compositions of the invention may be prepared in any suitable manner. For example, it may be prepared by blending together the protein, the carbohydrate source, and the fat source in appropriate proportions. If used, the emulsifiers may be included at this point. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently about 50° C. to about 80° C. to aid dispersal of the ingredients.

Commercially available liquefiers may be used to form the liquid mixture. The liquid mixture is then homogenised; for example in two stages.

The liquid mixture may then be thermally treated to reduce bacterial loads, by rapidly heating the liquid mixture to a temperature in the range of about 80° C. to about 150° C. for about 5 seconds to about 5 minutes, for example. This may be carried out by steam injection, autoclave or by heat exchanger; for example a plate heat exchanger.

Then, the liquid mixture may be cooled to about 60° C. to about 85° C.; for example by flash cooling. The liquid mixture may then be again homogenised; for example in two stages at about 10 MPa to about 30 MPa in the first stage and about 2 MPa to about 10 MPa in the second stage. The homogenised mixture may then be further cooled to add any heat sensitive components; such as vitamins and minerals. The pH and solids content of the homogenised mixture are conveniently adjusted at this point.

The homogenised mixture is transferred to a suitable drying apparatus such as a spray drier or freeze drier and converted to powder. The powder should have a moisture content of less than about 5% by weight. The probiotic bacterial strain may be added at this stage by dry-mixing. The lactoferrin may be added in the dry or in the wet phase.

If it is desired to produce a liquid composition, the mixture is filled into suitable containers, preferably aseptically. However, the composition may also be retorted in a container. Suitable apparatus for carrying out filling of this nature is commercially available. The liquid composition may be in the form of a ready-to-feed composition or in the form of a concentrate. The concentrate may also be administered directly to the infant or child, depending on the dosage required.

If it is desired to produce a powdered composition, the liquid mixture is preferably transferred to a suitable drying apparatus such as a spray drier or freeze drier and converted to powder. The powder should have a moisture content of less than about 5% by weight.

If the composition is a powder, it is preferably reconstituted in a liquid prior to administration to the infant and/or child.

The compositions of the invention may be adapted to infants and/or children of different ages. Additionally, they may be adapted to promote particular health benefits. Thus, by modifying at least one parameter selected from the list of lactoferrin concentration, probiotic concentration, nature of probiotic, caloric density, protein content, nature or quantity of prebiotic, the compositions of the invention may be tailored to specific needs and/or to particular age groups.

Thus, the composition of the invention may be administered to the infant or child at least once daily. It may also be administered at each feeding session and/or in between feeding sessions.

A kit of parts comprising a first composition according to the invention and a second composition according to the invention, wherein said first composition and said second composition differ by at least the presence of one nutrient or by the concentration of said nutrient in said composition also forms part of the invention. The first and second compositions are each adapted to fulfill the nutritional requirements in two different age groups.

Preferably, the first and second compositions differ by at least a parameter selected in the list comprising lactoferrin concentration, probiotic concentration, nature of probiotic, caloric density, protein content, nature or quantity of prebiotic.

The different composition of the kit of parts can each be adapted to be best suited for infants or children of specific age.

The kits of parts according to the invention can comprise 2, 3, 4, 5 ,6, 7, or more individual nutritional compositions—at least 2 of them being different (by their ingredients or the amount of each ingredients). Each individual nutritional composition can be targeted at delivering a specific benefit (i.e. to respond to a specific nutritional need of the specific target consumer).

In one embodiment a first composition is targeted at infants that are younger than the age targeted by the second composition.

In one embodiment, the caloric density of the first composition is between 55 to 75 kcal, or between 60 and 70 kcal, per 100 ml of composition (ready-to-feed liquid composition or dry powder reconstituted for feeding), preferably 61 and 65 kcal per 100 ml of composition.

The nutritional composition of the invention can be used for fulfilling at least partially the nutritional requirements of infants and/or children. Thus, it may be used as a supplement to the normal food intake of said infant or child. Alternatively, it may provide a full balanced meal to the infant or child.

In addition, the compositions of the invention can be used for providing health benefits in infants and/or children. The health benefits may include promoting gut maturation, enhancing gut health, enhancing protection later in life, contributing to support of natural defenses, contributing to support growth.

Thus, the compositions of the invention offer the advantages that they are suitable for infants and/or children for a number of health benefits. In addition the synergistic combination of probiotics and lactoferrin allows attaining health benefits in infants and/or children at a lower dosage than commonly used. With the compositions of the invention it is further possible to target a specific age group and/or a specific health benefit.

In one embodiment, the kit of parts of the invention comprises a first, a second, a third and optionally a fourth composition. Each of the compositions can be a composition as defined by any of claims 1 to 8 or 1 to 13 of the present patent application. Preferably the first and/or the second formulation are according to claims 1-8 or claims 1 to 13. At least 1 composition, preferably 2 or 3 compositions are according to the composition of claims 1-8 or 1 to 13. Preferably the first and second compositions differs (one from each other) by at least one of the following characteristics: the amount of probiotics, the type of the probiotics, the presence of lactoferrin, the amount of lactoferrin, the energy density, the amount of proteins, the amount of fatty acid, the nature of fatty acids.

In one embodiment the kit of parts is according to any of claims 15 to 19 and further comprises a third composition, optionally a fourth composition, wherein said third composition and/or said optional fourth composition each differ from said first and second composition by at least the presence of one nutrient or by the concentration of said nutrient in said composition, characterized in that said third and/or optionally fourth compositions are each adapted to fulfill the nutritional requirements in different age groups.

The points of differentiation of the compositions (nutritional requirements in different age groups) may be selected from at least one of the following: the amount of probiotics, the type of the probiotics, the presence of lactoferrin, the amount of lactoferrin, the energy density, the amount of proteins, the nature of the prebiotics, the amount of probiotics, the nature of oligosaccharides, the amount of oligosaccharides, the type of fatty acids, the amount of fatty acids.

Preferably one, 2 ,3 or all 4 of the compositions comprise at least one selective ingredient (and/or amount of ingredient) that is specifically beneficial to the infants at each targeted age of each compositions.

The present invention is further illustrated by means of the following non-limiting examples.

### Example 1

An example of the composition of an infant formula for use according to the present invention is given below. This composition is given by way of illustration only. The protein source is a mix of casein and whey protein (60% -40%). The below example can be intended for newly born infants.

| Nutrient | per l00 kcal | per litre |
|---|---|---|
| Energy (kcal) | 100 | 670 |
| Protein (g) | 1.83 | 12.3 |
| Fat (g) | 5.3 | 35.7 |
| Linoleic acid (g) | 0.79 | 5.3 |
| α-Linolenic acid (mg) | 101 | 675 |
| Lactose (g) | 11.2 | 74.7 |
| Prebiotic (100% GOS) (g) | 0.64 | 4.3 |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Lactoferrin (bovine) g | 0.149 | 1 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| I (μg) | 15 | 100 |
| Cu (mg) | 0.06 | 0.4 |
| Zn (mg) | 0.75 | 5 |

| | |
|---|---|
| Lactobacillus reuteri DSM | 2.10⁷ cfu/g of powder |
| 17938 | |
| (sourced from Biogaia) | |

### Example 2

The below example illustrates the kit of part of the invention with a first composition adapted for infants between 0 and 3 months and a second composition adapted for infants between 3 months and 6 months.

| | First composition | | Second composition, | |
|---|---|---|---|---|
| | for infants 0 to 3 months | | For infants 3 to 6 months | |
| Nutrient | per 100 kcal | per litre | per 100 kcal | per litre |
| Energy (kcal) | 100 | 670 | 100 | 630 |
| Protein (g) | 2.1 | 14.1 | 1.8 | 11.3 |
| Fat (g) | 5.3 | 35.7 | 5.0 | 31.5 |
| Linoleic acid (g) | 0.79 | 5.3 | 0.75 | 4.7 |
| α-Linolenic acid (mg) | 101 | 675 | 95 | 600 |
| Lactose (g) | 11.2 | 74.7 | 11.9 | 75 |
| Prebiotic (100% GOS) (g) | 0.64 | 4.3 | 0.63 | 4.0 |
| Minerals (g) | 0.37 | 2.5 | 0.37 | 2.3 |
| Na (mg) | 23 | 150 | 25 | 158 |
| K (mg) | 89 | 590 | 80 | 504 |
| Cl (mg) | 64 | 430 | 65 | 410 |
| Ca (mg) | 62 | 410 | 60 | 378 |
| P (mg) | 31 | 210 | 33 | 208 |
| Mg (mg) | 7 | 50 | 7 | 44 |
| Mn (μg) | 8 | 50 | 5 | 32 |
| Se (μg) | 2 | 13 | 3 | 19 |
| Vitamin A (μg RE) | 105 | 700 | 90 | 570 |
| Vitamin D (μg) | 1.5 | 10 | 1.5 | 9.5 |
| Vitamin E (mg TE) | 0.8 | 5.4 | 0.8 | 5.0 |
| Vitamin K1 (μg) | 8 | 54 | 8 | 50 |
| Vitamin C (mg) | 10 | 67 | 15 | 95 |
| Vitamin B1 (mg) | 0.07 | 0.47 | 0.1 | 0.6 |
| Vitamin B2 (mg) | 0.15 | 1.0 | 0.1 | 0.6 |
| Niacin (mg) | 1 | 6.7 | 0.5 | 3.2 |
| Vitamin B6 (mg) | 0.075 | 0.50 | 0.06 | 0.4 |
| Lactoferrin (bovine) g | 0.15 | 1.0 | 0.05 | 0.3 |
| Folic acid (μg) | 9 | 60 | 15 | 95 |
| Pantothenic acid (mg) | 0.45 | 3 | 0.8 | 5.0 |
| Vitamin B12 (μg) | 0.3 | 2 | 0.2 | 1.3 |
| Biotin (μg) | 2.2 | 15 | 2.0 | 12.6 |
| Choline (mg) | 10 | 67 | 15 | 95 |
| Fe (mg) | 1.2 | 8 | 1.0 | 6.3 |
| I (μg) | 15 | 100 | 15 | 95 |
| Cu (mg) | 0.06 | 0.4 | 0.06 | 0.4 |
| Zn (mg) | 0.75 | 5 | 0.9 | 5.7 |

| | | |
|---|---|---|
| Lactobacittus reuteri DSM | 2.10⁴ cfu/g of powder | 2.10⁷ cfu/g of powder |
| 17938 | | |
| (sourced from Biogaia) | | |

### Example 3

An example of the composition of a follow up infant formula for use according to the present invention is given below. The composition is targeted at infant 6 to 12 months. This composition is given by way of illustration only. Importantly, the below composition(s) can be combined with the composition of examples 1 or 2 in order to make up kits of parts according to the invention. These kits of parts provide better nutrition characteristics for each individual age, in response to specific nutritional needs. The proteins of the below composition are from whey and casein in a ratio of 50/50. In an alternative, the proteins are from whey only or 70% (w/w) from whey. In the below example 16% (w/w) of the carbohydrates are rice carbohydrates (in a similar alternative example 25% of the carbohydrates are rice carbohydrates). In an alternative example, the carbohydrate are primarily from milk and comprise lactose and maltodextrine only (with a very low level of starch—below 1%, 5% or 10% w/w). Prebiotics can be added (e.g. GOS, 0.5 g/100 kcal) to the formulations.

| | Follow up infant formula | |
|---|---|---|
| | For infants 6 to 12 months | |
| Nutrient | per l00 kcal | per litre |
| Energy (kcal) | 100 | 670 |
| Protein (g) | 2.2 | 14.6 |
| Fat (g) | 3.7 | 31.6 |
| Linoleic acid (g) | 0.76 | 5.1 |
| α-Linolenic acid (mg) | 88 | 590 |
| Lactose (g) | 7.44 | 81.2 |
| Maltodextrine (g) | 2.1 | 14.2 |
| Starch (g) | 2.59 | 17.3 |
| Prebiotic (100% GOS) (g) | 0.63 | 4.0 |
| Minerals (g) | 0.58 | 3.9 |
| Na (mg) | 39 | 158 |
| K (mg) | 113 | 760 |
| Cl (mg) | 73 | 490 |
| Ca (mg) | 105 | 700 |
| P (mg) | 66 | 440 |
| Mg (mg) | 11.4 | 76 |
| Vitamin A (μg RE) | 90 | 570 |
| Vitamin D (μg) | 1.5 | 9.5 |
| Vitamin E (mg TE) | 0.8 | 5.0 |
| Vitamin K1 (μg) | 8 | 50 |
| Vitamin C (mg) | 15 | 95 |
| Vitamin B1 (mg) | 0.1 | 0.6 |
| Vitamin B2 (mg) | 0.1 | 0.6 |
| Niacin (mg) | 0.5 | 3.2 |
| Vitamin B6 (mg) | 0.06 | 0.4 |
| Lactoferrin (bovine) | 0.025 | 0.15 |
| Folic acid (μg) | 15 | 95 |
| Pantothenic acid (mg) | 0.8 | 5.0 |
| Vitamin B12 (μg) | 0.2 | 1.3 |
| Biotin (μg) | 2.0 | 12.6 |
| Choline (mg) | 15 | 95 |
| Fe (mg) | 1.0 | 6.3 |
| I (μg) | 15 | 95 |
| Cu (mg) | 0.06 | 0.4 |
| Zn (mg) | 0.9 | 5.7 |

| | |
|---|---|
| Lactobacillus reuteri DSM 17938 | 10⁷ cfu/g of powder |
| (sourced from Biogaia - BioGaia AB, | |
| Sweden) | |
| AND/OR | AND/OR |
| Lactobacillus rhamnosus CGMCC | 10⁶ cfu/g of powder |
| 1.3724 | |
| AND/OR | AND/OR |
| BB536 (=ATCC BAA-999, deposited by | 10⁶ cfu/g of powder |
| Morinaga, (sourced from Morinaga Milk | |
| Industry, Co., Ltd, Tokyo, Japan) | |

1. A nutritional composition for infants and/or children comprising lactoferrin and probiotics.

2. A nutritional composition according to claim 1, wherein the lactoferrin is selected from the group consisting of human colostrum lactoferrin, human milk lactoferrin, bovine colostrum lactoferrin, and bovine milk lactoferrin.

3. A nutritional composition according to claim 1, wherein the probiotic is selected from the group consisting of a lactobacillus, a bifidobacterium, an Enterococcus, a saccharomyces or a streptococcus, preferably from Lactobacillus rhamnosus, Lactobacillus paracasei, Lactobacillus reuteri, Lactobacillus acidophilus, Lactobacillus johnsonii, Lactobacillus plantarum, Lactobacillus salivarius, Streptococcus thermophilus, Bifidobacterium longum, Bifidobacterium lactis, Bifidobacterium breve, Enterococcus faecium, Streptococcus sp. and Saccharomyces boulardii and mixtures thereof.

4. A nutritional composition according to claim 1, wherein the amount of lactoferrin is between 8 g and 0.1 g per liter of ready-to feed liquid composition or reconstituted dry powder.

5. A nutritional composition according to claim 1, wherein the amount of probiotic is between 10² and 10⁹ colony forming unit (cfu) per g of dry composition.

6. A nutritional composition according to claim 1, wherein the composition comprises additional ingredients selected from the group consisting of prebiotics, preferably N-acetylated oligosaccharide, sialylated oligosaccharide, fucosylated oligosaccharide, galacto-oligosaccharides, fructo-oligosaccharide or mixtures thereof, vitamins, minerals, carbohydrates, fatty acids, lipids, protein, and may-mixtures thereof.

7. A nutritional composition according to claim 1, the composition being in a form selected from the group consisting of a powder and a liquid.

8. A nutritional composition according to claim 1, the composition having a caloric density of between 60 to 70 kcal per 100 ml of composition.

9. A method for fulfilling at least partially the nutritional requirements of infants and/or children comprising the steps of providing a nutritional composition for infants and/or children comprising lactoferrin and probiotics to the infant and/or child.

10. A method for use in providing health benefits in infants and/or children comprising the steps of providing a nutritional composition for infants and/or children comprising lactoferrin and probiotics to the infant and/or child.

11. The method according to claim 10, wherein the infants and/or children are of 0 months to 6 years of age.

12. A nutritional composition of claim 1 wherein the caloric density is adapted for the age of the target child or infant, preferably between 62 and 67 kcal/100 ml for infants between birth and 2 months, between 61 and 65 kcal/100 ml for infants between 3 months and 12 months, and below 63 kcal/100 ml for children old than 12 months.

13. The method according to claim 10, wherein the health benefits are selected from the group consisting of promoting gut maturation, promoting enteric and/or neuronal maturation and/or development, enhancing gut health, enhancing gut comfort, reducing colics, reducing gut pain, enhancing cognitive development, enhancing protection later in life, contributing to support of natural defenses, contributing to support growth, and fulfilling at least partially the nutritional requirements of the infant and/or child.

14. A method for preparing a nutritional composition comprising lactoferrin and probiotics comprising the steps of mixing both while preserving a high bioavailability of said lactoferrin and a high survival rate of said probiotics.

15. A kit of parts comprising a first composition for infants and/or children comprising lactoferrin and probiotics and a second composition wherein the first composition and the second composition differ by at least the presence of one nutrient or by the concentration of the nutrient in the composition, the first and second compositions are each adapted to fulfill the nutritional requirements of two different age groups.

16. A kit according to claim 15, wherein the first composition and the second composition differ by at least a parameter selected from the group consisting of lactoferrin concentration, probiotic concentration, nature of probiotic, caloric density, protein content, and nature or quantity of prebiotic.

17. A kit of claim 15 wherein the first composition has a caloric density greater than the caloric density of the second composition.

18. A kit of claim 15 wherein the first composition is adapted to infants of a first age and the second composition is adapted to infants of a second age and wherein the first age is younger than the second age.

19. A kit of claim 15 wherein the amount of lactoferrin in the first and second composition is adapted for the specific age of the infant.

20. A kit of parts according to claim 15 further comprising a third composition, wherein the third composition differs from the first and second composition by at least the presence of one nutrient or by the concentration of the nutrient in the composition, the third composition is adapted to fulfill the nutritional requirements in different age groups.

21. A nutritional composition according to claim 1, wherein the amount of lactoferrin is between 1500 mg and 500 mg per 100 g of dry composition (w/w).
